(19) United States
(12) Patent Application Publication
(10) Pub. No.: US 2016/0317791 A1
(43) Pub. Date: Nov. 3, 2016

(54) SINUS TREATMENT

(71) Applicant: Rainbow Medical Ltd., Herzliya (IL)

(72) Inventors: Yossi Gross, Moshav Mazor (IL); Hadar Better, Tel Aviv (IL)

(21) Appl. No.: 14/701,474

(22) Filed: Apr. 30, 2015

(51) Int. Cl. A61M 25/10; A61B 1/233; A61M 31/00

(52) U.S. Cl. CPC A61M 25/1011; A61M 31/00; A61B 1/233; A61M 2025/1052; A61M 2210/0681

(57) ABSTRACT

Apparatus and methods are described, including apparatus for treating a maxillary sinus of a subject. The apparatus includes a tube shaped to define a lumen thereof, a lateral wall of the tube at a distal portion of the tube being shaped to define an aperture. A first radially-deployable seal is coupled to the tube proximally to the aperture, and a second radially-deployable seal is coupled to the tube distally to the aperture. Each of the first and second seals is configured to, when radially deployed inside a nasal cavity of the subject, generally prevent flow from one side of the seal to another side of the seal of fluid passing out of the aperture. Other applications are also described.

### CROSS REFERENCE TO RELATED APPLICATIONS

The present application is related to an application entitled “Sinus Treatment” to Gross et al., filed on even date herewith.

### FIELD OF THE INVENTION

Applications of the present invention relate generally to treatment of sinus disorders, and specifically to methods and apparatus for opening a natural ostium of a sinus.

### BACKGROUND

Sinusitis is a common condition, characterized by symptoms such as nasal discharge, facial and ear pressure and pain, headache, loss of smell, fever, cough and fatigue. Physiological clearing of paranasal sinuses is via mucociliary transport through the ostia (natural sinus openings into a nasal cavity). Disruption of this function, often associated with partial blockage of an ostium, allows stagnation of mucous secretions and alteration of pH and other physiologic parameters, making the sinus conditions more favorable to microbiological growth and, therefore, susceptible to sinusitis. Treatment of chronic sinusitis typically includes antibiotics, steroids, decongestants and, in some cases, surgical procedures such as lavage.

U.S. 2013/0030545 to Gross et al., which is incorporated herein by reference, describes paranasal sinus apparatus, including a hole-forming member configured to form a hole through a bone wall of a paranasal sinus of a subject. An implant is provided, having a proximal end and a distal end, and which is shaped to define a lumen. The implant is couplable to the hole-forming member, and is securable to the wall and extendable through the hole, such that the proximal end is disposed external to the paranasal sinus and the distal end is disposed within the paranasal sinus. The implant includes a biodegradable material.

### SUMMARY OF THE INVENTION

Applications of the present invention include a method for opening a natural ostium of a maxillary sinus of a subject. A tube is inserted into the subject's nasal cavity, and is aligned with respect to the sinus such that an aperture in a wall of the tube is aligned generally opposite to the ostium. Subsequently, one or more seals are deployed near the aperture. For example, a first balloon may be inflated proximally to the aperture, and a second balloon may be inflated distally to the aperture. The deployment of the seals fluidly isolates the portion of the nasal cavity that is adjacent to the ostium. Subsequently, a stream of fluid is passed out of the aperture and into the fluidly isolated portion of the nasal cavity. The pressure of the fluid increases, until the ostium is opened.

There is therefore provided, in accordance with some applications of the present invention, apparatus for treating a maxillary sinus of a subject, the apparatus including;

a tube shaped to define a lumen thereof, a lateral wall of the tube at a distal portion of the tube being shaped to define an aperture;

a first radially-deployable seal, coupled to the tube proximally to the aperture; and

a second radially-deployable seal, coupled to the tube distally to the aperture,
each of the first and second seals being configured to, when radially deployed inside a nasal cavity of the subject, generally prevent flow from one side of the seal to another side of the seal of fluid passing out of the aperture.

In some applications, at least one of the first and second seals is an annular seal.

In some applications, both of the first and second seals are annular seals.

In some applications, at least one of the first and second seals includes a balloon.

In some applications, the first seal includes a first balloon and the second seal includes a second balloon.

In some applications, respective interiors of the first and second balloons are permanently in fluid communication with each other.

In some applications, at least one of the first and second seals includes a flap.

In some applications, the second seal includes a flap.

In some applications, the apparatus further includes an introducer sheath shaped to cover the flap, and the flap is configured to be radially deployed by uncovering the flap by withdrawing the introducer sheath.

In some applications, an outside diameter of the first seal is 3-10 mm, upon the first seal being maximally radially deployed.

In some applications, an outside diameter of the second seal is 3-10 mm, upon the second seal being maximally radially deployed.

In some applications, an outside diameter of the first seal is at least 10% larger than an outside diameter of the second seal, upon the first and second seals being maximally radially deployed.

In some applications, the outside diameter of the first seal is less than 100% larger than the outside diameter of the second seal, upon the first and second seals being maximally radially deployed.

In some applications, the apparatus further includes a deflector disposed within the lumen of the tube, the deflector being shaped to deflect out of the aperture a wire that is advanced distally within the lumen of the hollow tube.

In some applications, the deflector includes an appendage extending from an inside wall of the tube.

In some applications, the deflector includes a curved channel.

In some applications, the apparatus further includes an imaging device coupled to the distal portion of the tube.

In some applications, at least a portion of the tube is transparent.

In some applications, a distance between the first and second seals, upon the first and second seals being maximally radially deployed, is 10-30 mm.

In some applications, a diameter of the aperture is 1-5 mm.

In some applications, a length of the tube is 2-30 cm.

In some applications, an outer diameter of the tube is 2-6 mm.

In some applications, the tube is placeable over a distal end of an endoscope.

In some applications, the apparatus further includes the endoscope.

There is further provided, in accordance with some applications of the present invention, a method for treating a natural ostium of a maxillary sinus of a subject, the method including:

opening the ostium, by pumping fluid into a nasal cavity of the subject such that the pumped fluid is in direct contact with the nasal cavity and reaches a maximum pressure; and

subsequently, rinsing the sinus, by pumping rinsing fluid into the sinus at a pressure that is less than the maximum pressure.

In some applications, rinsing the sinus includes using a syringe to alternatingly (i) pump the rinsing fluid into the sinus, and (ii) not pump any fluid into the sinus.

In some applications, using the syringe includes using the syringe to alternatingly (i) pump the rinsing fluid into the sinus, and (ii) draw fluid from the sinus.

In some applications,

the syringe includes a first channel and a second channel,

pumping the rinsing fluid into the sinus includes pumping the rinsing fluid into the sinus through the first channel, and

drawing fluid from the sinus includes drawing fluid from the sinus through the second channel.

In some applications, the method further includes forming an artificial hole in a bone wall of the sinus, and rinsing the sinus includes causing the rinsing fluid to pass out of the sinus through the artificial hole.

There is further provided, in accordance with some applications of the present invention, a method for treating a natural ostium of a maxillary sinus of a subject, the method including;

inserting a tube into a nasal cavity of the subject, a wall of the tube at a distal portion of the tube being shaped to define an aperture;

subsequently, moving the tube such that the aperture is aligned generally opposite to the ostium;

subsequently, fluidly isolating a portion of the nasal cavity that is adjacent to the ostium from (a) a lung of the subject, and (b) an outer opening of a nostril of the subject, by deploying one or more seals near the aperture; and

subsequently, opening the ostium, by passing a stream of fluid out of the aperture and into the fluidly-isolated portion of the nasal cavity.

In some applications, the method further includes, before inserting the tube:

performing a computed tomography (CT) scan on the subject;

in response to anatomical detail shown in the CT scan, identifying a desired property of one or more of the seals; and

causing the seals to be manufactured such that the seals have the desired property.

In some applications, the desired property is selected from the group consisting of: a shape, and a diameter, the method including identifying the desired property.

In some applications, the method further includes, before inserting the tube:

performing a computed tomography (CT) scan on the subject; and

in response to anatomical detail shown in the CT scan, selecting one or more of the seals from a plurality of seals that differ from each other in a particular property.

In some applications, the particular property is selected from the group consisting of: a shape, and a diameter, the method including selecting one or more of the seals from a plurality of seals that differ from each other in the selected property.

In some applications, moving the tube such that the aperture is aligned generally opposite to the ostium includes aligning the aperture with respect to the ostium by using an imaging device disposed within the nasal cavity of the subject.

In some applications, moving the tube such that the aperture is aligned generally opposite to the ostium includes aligning the aperture with respect to the ostium by (i) passing a guidewire out of the aperture, and (ii) probing tissue of the nasal cavity with the guidewire.

In some applications, deploying the one or more seals includes deploying one or more annular seals.

In some applications, passing the stream of fluid includes passing a stream of saline.

In some applications, the method further includes:

using an imaging device to view the ostium while passing the stream of fluid out of the aperture; and

stopping to pass the stream of fluid, upon viewing, using the imaging device, that the ostium has been opened.

In some applications, the method further includes, before inserting the tube into the nasal cavity of the subject, forming an artificial hole in a bone wall of the sinus.

In some applications, the method further includes stopping to pass the stream of fluid in response to fluid exiting a nose of the subject.

In some applications, at least one of the seals includes a balloon, and deploying the seals includes inflating the balloon.

In some applications, deploying the one or more seals includes radially deploying (a) a first seal, coupled to the tube proximally to the aperture, and (b) a second seal, coupled to the tube distally to the aperture.

In some applications, radially deploying the first and second seals includes radially deploying the first and second seals by passing the stream of fluid out of the aperture.

In some applications,

the first seal includes a first flap,

the second seal includes a second flap, and

radially deploying the first and second seals includes radially deploying the first and second flaps by causing the fluid to apply a force to the first and second flaps.

In some applications,

the first seal includes a first balloon,

the second seal includes a second balloon, and

radially deploying the seals includes inflating the first and second balloons.

In some applications, inflating the first and second balloons includes (a) inflating the first balloon to a first diameter, and (b) inflating the second balloon to a second diameter that is less than the first diameter.

In some applications, inflating the first and second balloons includes (a) inflating the first balloon to a first pressure, and (b) inflating the second balloon to a second pressure that is less than the first pressure.

In some applications, inflating the first and second balloons includes inflating the first and second balloons to a single pressure.

In some applications, inflating the first and second balloons includes:

using a syringe that includes a pressure sensor to inflate the first and second balloons; and

inflating the first and second balloons until the pressure sensor detects that respective internal pressures of the first and second balloons have reached respective thresholds.

In some applications, the syringe is configured to automatically stop inflating the first and second balloons in response to the respective internal pressures reaching the respective thresholds, the method including using the syringe to inflate the first and second balloons.

In some applications, the respective thresholds are different from one another, the method including inflating the first and second balloons until the pressure sensor detects that the respective internal pressures have reached the respective thresholds.

In some applications, the respective thresholds are equal to one another, the method including inflating the first and second balloons until the pressure sensor detects that the respective infernal pressures have reached the respective thresholds.

In some applications, at least one of the seals includes a flap, and deploying the seals includes radially deploying the flap by uncovering the flap.

In some applications, the method further includes, following the opening of the ostium, passing a tool through the tube and into the ostium.

In some applications, the tool includes a guidewire, the method including passing the guidewire into the ostium.

In some applications, the fool includes a balloon, the method further including inflating the balloon within the ostium.

In some applications, the tool includes a rinsing tube, the method further including using the rinsing tube to rinse the sinus.

In some applications,

the rinsing tube is shaped to define a first tube lumen and a second, tube lumen,

using the rinsing tube to rinse the sinus includes passing fluid through the first tube rumen, and

the method further includes draining the sinus through the second tube lumen.

In some applications, the tool includes an imaging device, the method further including using the imaging device to image an interior of the sinus.

In some applications,

inserting the tube includes inserting the tube while the tube surrounds a distal end of an endoscope, and

moving the tube includes moving the tube while the tube surrounds the distal end of the endoscope.

In some applications, the method further includes, before opening the ostium, withdrawing the endoscope without withdrawing the tube.

There is further provided, in accordance with some applications of the present invention, apparatus for treating a maxillary sinus of a subject, the apparatus including:

a tube shaped to define a lumen thereof, a lateral wall of the tube at a distal portion of the tube being shaped to define an aperture; and

exactly one balloon coupled to the tube, the balloon being configured to, when inflated inside a nasal cavity of the subject, generally prevent flow of fluid that passes out of the aperture from a portion of the nasal cavity that is adjacent to the ostium (a) to a lung of the subject, and (b) to an outer opening of a nostril of the subject.

The present invention will be more fully understood from the following detailed description of applications thereof, taken together with the drawings, in which:

### BRIEF DESCRIPTION OF THE DRAWINGS

FIG. 1 is a schematic illustration of a method for treating a natural ostium of a maxillary sinus of a subject, in accordance with some applications of the present invention;

FIGS. 2A-B and 3A-E are schematic illustrations of apparatus for treating a maxillary sinus of a subject, in accordance with some applications of the present invention;

FIGS. 4-B are schematic illustrations of a radial deployment of a flap, in accordance with some applications of the present invention;

FIGS. 5A-B show cross-sections of the distal portion of apparatus for treating a maxillary sinus of a subject, in accordance with some applications of the present invention;

FIG. 6 is a schematic illustration of a tube placed over the distal end of an endoscope, in accordance with some applications of the present invention;

FIG. 7 is an illustrative plot showing the pressure of fluid that is passed from an aperture during treatment of a natural ostium, in accordance with some applications of the present invention;

FIG. 8 is a schematic illustration showing the passing of a tool into an ostium, in accordance with some applications of the present invention; and

FIGS. 9A-B are schematic illustrations of apparatus for treating a maxillary sinus of a subject, in accordance with some applications of the present invention.

### DETAILED DESCRIPTION OF APPLICATIONS

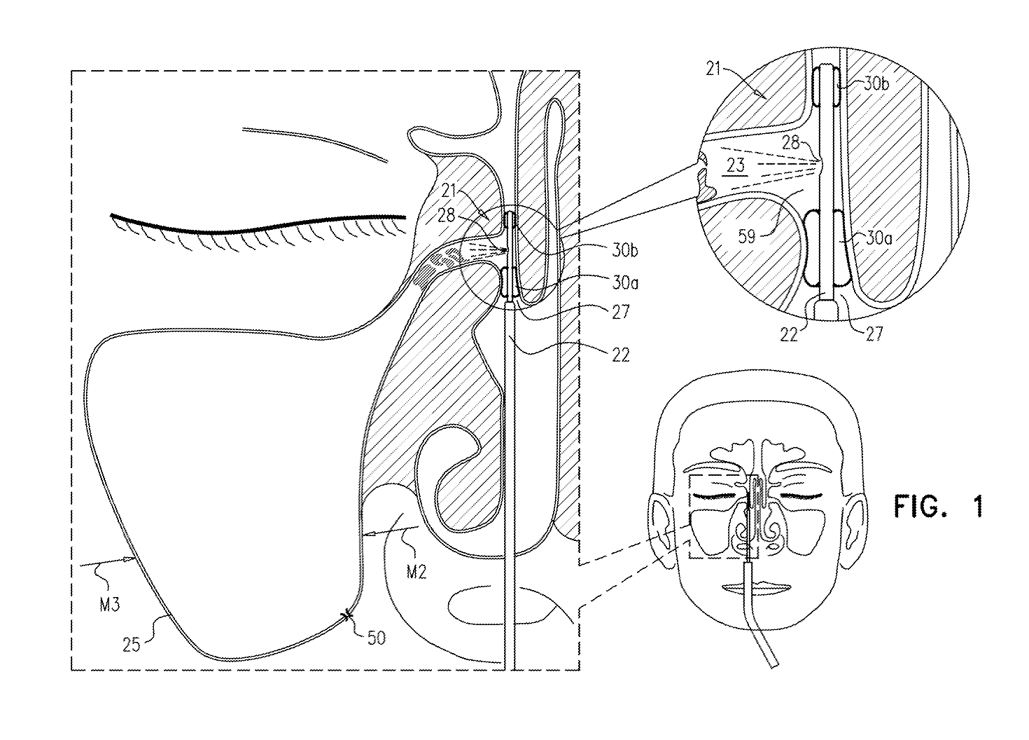

Reference is made to FIG. 1, which is a schematic illustration of a method 21 for treating a natural ostium 23 of a maxillary sinus 25 of a subject, in accordance with some applications of the present invention. In method 21, a tube 22 is inserted into a nasal cavity 27 of the subject, typically while the subject is under a local anesthetic. The tube is moved such that an aperture 28 in the tube wall is aligned generally opposite to the ostium. Subsequently, one or more seals (e.g., two seals 30a and 30b) are deployed near the aperture. By deploying the seals, the portion 52 of the nasal cavity that is adjacent to the ostium is fluidly isolated from (a) the subject's lungs (which are distal to the ostium), and (b) an outer opening of the subject's nostril (which is proximal to ostium). Next, a stream of fluid (e.g., saline) is passed out of the aperture and into portion 59 of the nasal cavity. The pressure of the fluid increases within the fluidly-isolated portion of the nasal cavity, until the pressure of the fluid causes the ostium to open. In some applications, pulsatile pressure is applied to the ostium, by passing the fluid out of the aperture in a pulsatile manner.

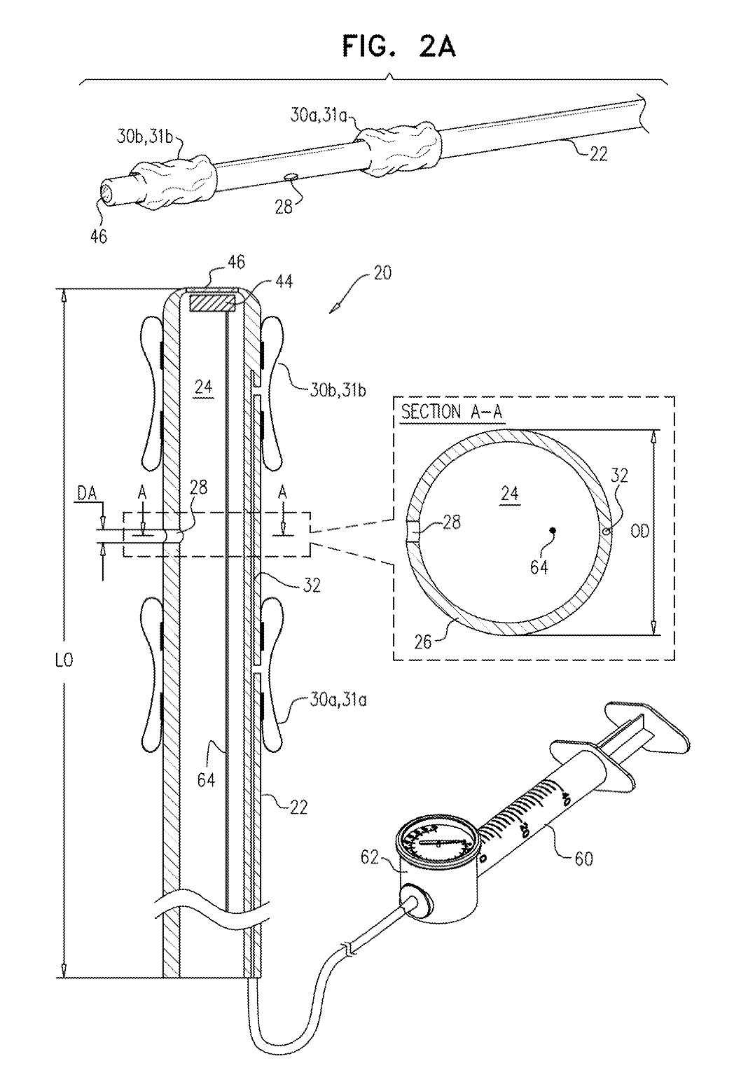

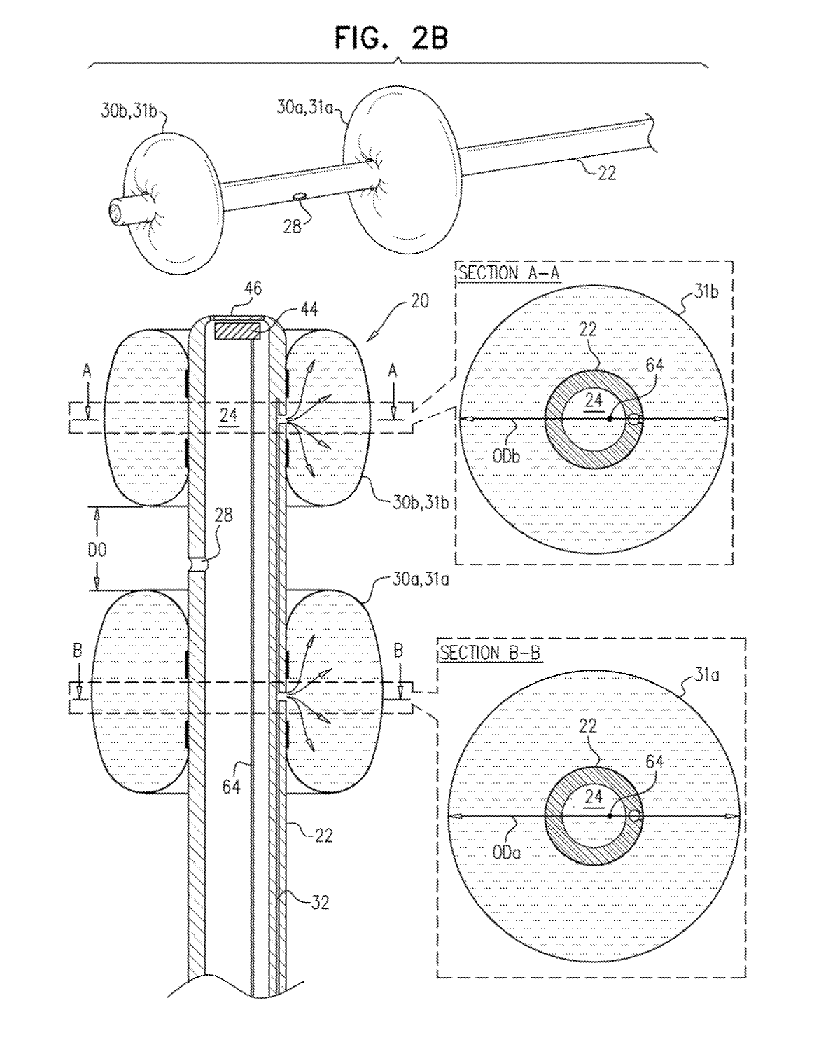

Reference is now made to FIGS. 2A-B and 3A-B, which are schematic illustrations of apparatus 20 for treating a maxillary sinus of a subject, in accordance with some applications of the present invention. Apparatus 20 may be used to perform method 21, described hereinabove with reference to FIG. 1. (Some aspects of apparatus 20 were at least partly described above, with reference to FIG. 1.)

Apparatus 20 comprises tube 22, shaped to define a lumen 24 thereof. A lateral wall 26 of tube 22 at a distal portion of the tube is shaped to define an aperture 28, which, in some applications, has a diameter DA of 1-5 mm. (In other applications, diameter DA is less than 1 mm or greater than 5 mm. In yet other applications, aperture 28 is not circular, but is instead of a different shape, e.g., aperture 28 may be a horizontal or vertical slit.) Apparatus 20 further comprises a first radially-deployable seal 30a, coupled to the tube proximally to aperture 26, and a second radially-deployable seal 30b, coupled to the tube distally to the aperture. As described hereinabove with reference to FIG. 1, each of the first and second seals 30a and 30b is configured to, when radially deployed inside the nasal cavity, generally prevent flow, from one side of the seal to another side of the seal, of the fluid that is passed out of the aperture. The length L0 of the tube is typically at least 2 cm and/or less than 30 cm, e.g., 4-20 cm. The outer diameter OD of the tube is typically at least 2 mm and/or less than 6 mm. In some applications, tube 22 is steerable.

Typically, at least one of the first and second seals is an annular seal, e.g., both of the seals may be annular seals. In some applications, at least one of the first and second seals comprises a balloon. For example, as shown in FIGS. 2A-B, the first seal may comprise a first balloon 31a, and the second seal may comprise a second balloon 31b. In some applications, respective interiors of the first and second balloons are permanently in fluid communication with each other. For example, a single inflation tube 32 passing in a proximal-distal direction through lateral wall 26 may be connected to both of the balloons, such that each one of the balloons is inflated to the same pressure as the other one of the balloons. In other applications, the balloons are inflated separately (via separate inflation tubes), to different respective pressures. Since the first balloon has a greater surface area facing portion 59 (FIG. 1) of the nasal cavity than does the second balloon, the first balloon is subjected to a greater force from fluid within portion 59 of the nasal cavity than is the second balloon. Hence, the first balloon may be inflated to a pressure that is greater than the pressure to which the second balloon is inflated. (The greater internal pressure of the first balloon allows for a greater frictional force between the first balloon and the wall, which allows the first balloon to withstand the force from the fluid, i.e., to remain in place despite the force from the fluid.)

Typically, the balloons are inflated with a fluid (i.e., a gas, such as air, and/or a liquid, such as saline), e.g., using a syringe 60. In some applications, the syringe includes a pressure sensor (e.g., a mechanical pressure gauge 62), and the first and second balloons are inflated until the pressure sensor detects that respective internal pressures of the first and second balloons have reached respective thresholds. (As described hereinabove, the respective thresholds may be different from one another, or alternatively, equal to one another.) In some applications, the syringe automatically stops inflating the first and second balloons in response to the respective internal pressures reaching the respective thresholds.

FIG. 2A shows balloons 31a and 31b in an undeployed (deflated) state, while FIG. 2B shows the balloons in a maximally-radially-deployed (maximally inflated) state. Upon being maximally radially deployed, the outside diameter ODa of the first seal is typically at least 3 and/or less than 10 mm, 3-10 mm being a typical range for the inner diameter of the nasal cavity proximally to the ostium. Upon being maximally radially deployed, the outside diameter ODb of the second seal is also typically at least 3 and/or less than 10 mm, 3-10 mm also being a typical range for the inner diameter of the nasal cavity distally to the ostium. Typically, however, ODa is at least 10% larger (but less than 100% larger) than ODb, since the inner diameter of the nasal cavity proximally to the ostium is typically at least 10% larger (but less than 100% larger) than the inner diameter of the nasal cavity distally to the ostium.

In some applications, the first and second seals are deployed to their maximum deployment diameters ODa and ODb. In other applications, at least one of the seals is not deployed to its maximum deployment diameter. In any case, upon being deployed, the outside diameter of the first seal is typically larger (e.g., 10%-100% larger) than that of the second seal, since, as noted above, the inner diameter of the nasal cavity is typically greater proximally to the ostium than distally to the ostium. In other words, typically, the first balloon is inflated to a first diameter (e.g., ODa), and the second balloon is inflated to a second diameter (e.g., ODb) that is less than the first diameter. Typically, apparatus 20 does not comprise circuitry to inflate the balloons repeatedly in alternation.

Typically, the distance D0 between the first and second seals, upon the first and second seals being maximally radially deployed, is at least 10 mm and/or less than 30 mm. D0 is typically fixed for a given tube, i.e., it cannot be changed without disassembling the device. D0 is large enough such that the seals do not prevent the fluid from being passed out of the aperture, yet D0 is also small enough such that the ostium may be opened quickly and efficiently, i.e., only a relatively small amount of fluid needs to be passed out of the tube before the ostium is opened, since the size of fluidly-isolated portion 59 is not too large.

In some applications, as shown in FIGS. 2A-B and 3A-B, aperture 23 is supplied by lumen 24 of tube 22. In other applications, a separate fluid-delivery tube, e.g., a fluid-delivery tube passing longitudinally through the wall of tube 22, supplies fluid to aperture 28. Typically, a syringe or electromechanical pump is used to supply the fluid to the aperture. In some applications, the syringe or electromechanical pump that is used to inflate first balloon 31a and/or second balloon 31b (e.g., syringe 60) also supplies the fluid to the aperture.

In some applications, apparatus 20 further comprises an imaging device 44 (e.g., a camera or fiber-optic array) coupled to the distal portion of the tube. Imaging device 44 facilitates the advancement of the tube inside the nasal cavity, by providing images of the nasal cavity as the tube is advanced. To facilitate the imaging, at least a portion of the tube (e.g., the distal wall 46 of the tube) may be transparent. The images may be transmitted wirelessly, or via a wire 64, as shown in FIG. 2A.

In some applications, as shown in FIGS. 3A-B, at least one of the first and second seals comprises a flap. For example, as shown in FIGS. 3A-B, the first seal may comprise a first flap 34a, and the second seal may comprise a second flap 34b, Alternatively, only one of the seals (e.g., the second seal) comprises a flap. FIG. 3A shows flaps 34a and 34b in an undeployed state, while FIG. 3B shows the flaps in a radially-deployed state. As shown in FIG. 3B, the seals may be radially deployed by passing the stream of fluid out of the aperture. For example, for flaps 34a and 34b, the stream of fluid may be used to apply a force to the flaps that causes the flaps to be radially deployed. In general, the dimensions (e.g., diameters) described above with respect to balloons 31a and 31b are also relevant to flaps 34a and 34b.

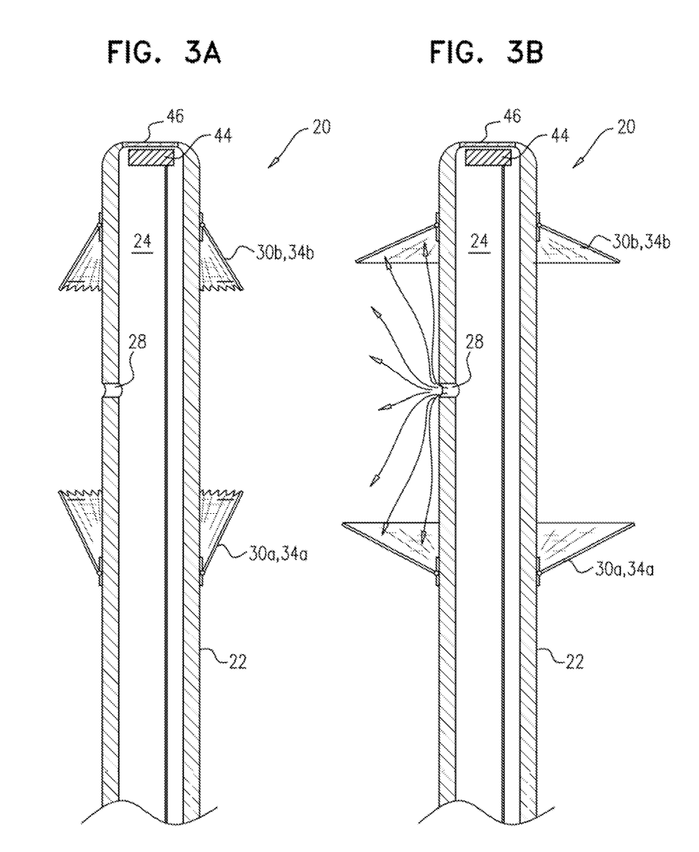

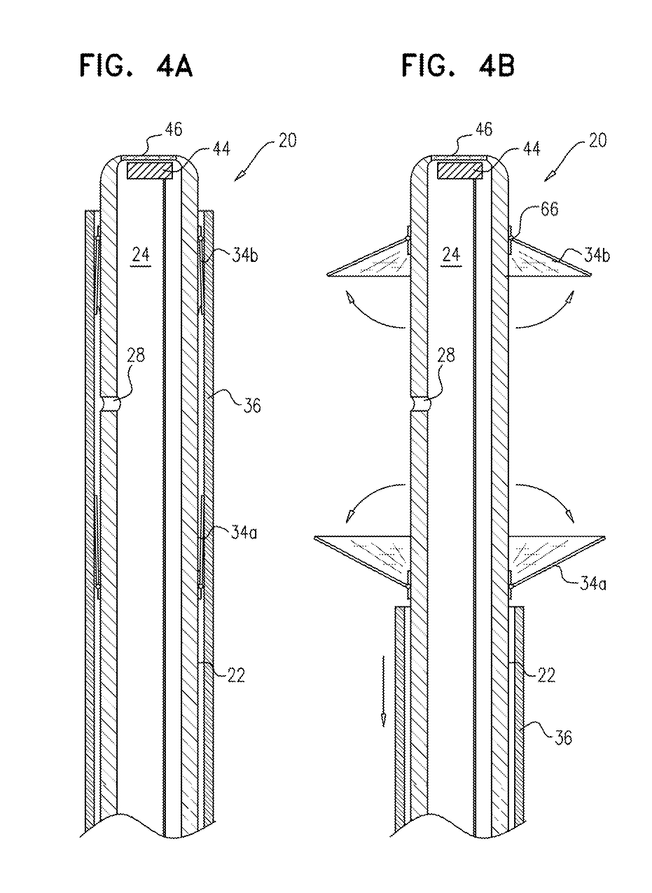

Reference is now made to FIGS. 4A-B, which are schematic illustrations of a radial deployment of a flap, in accordance with some applications of the present invention, In some applications, at least one of the flaps is radially deployed, by being uncovered. For example, in some applications, apparatus 20 further comprises an introducer sheath 36 shaped to cover one or more of the flaps, and the flaps are radially deployed within the nasal cavity by withdrawing the introducer sheath. FIGS. 4A-B (as well as FIGS. 3A-B) show flaps 34a and 34b connected to the tube via spring hinges 66. When the introducer tube is withdrawn, hinges 66 spring open, thus deploying the flaps. it is noted, however, that the flaps need not necessarily be connected to the tube via dedicated hinges. Rather, the flaps themselves may act as springs, such that the flaps store potential energy when covered by the introducer sheath, and spring open upon the introducer sheath being withdrawn. In some applications, the flaps are made of a shape-memory material, which facilitates the springing-open of the flaps.

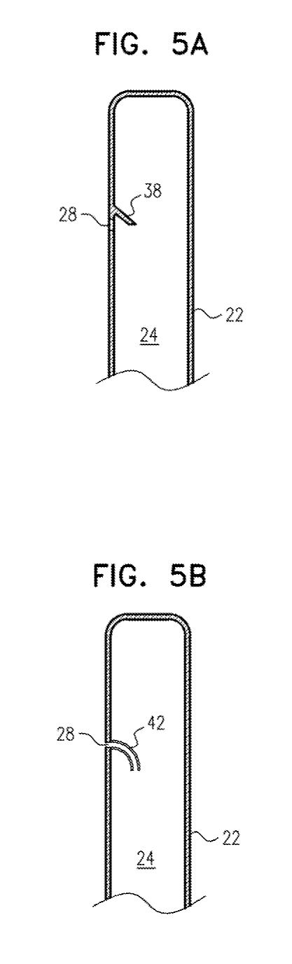

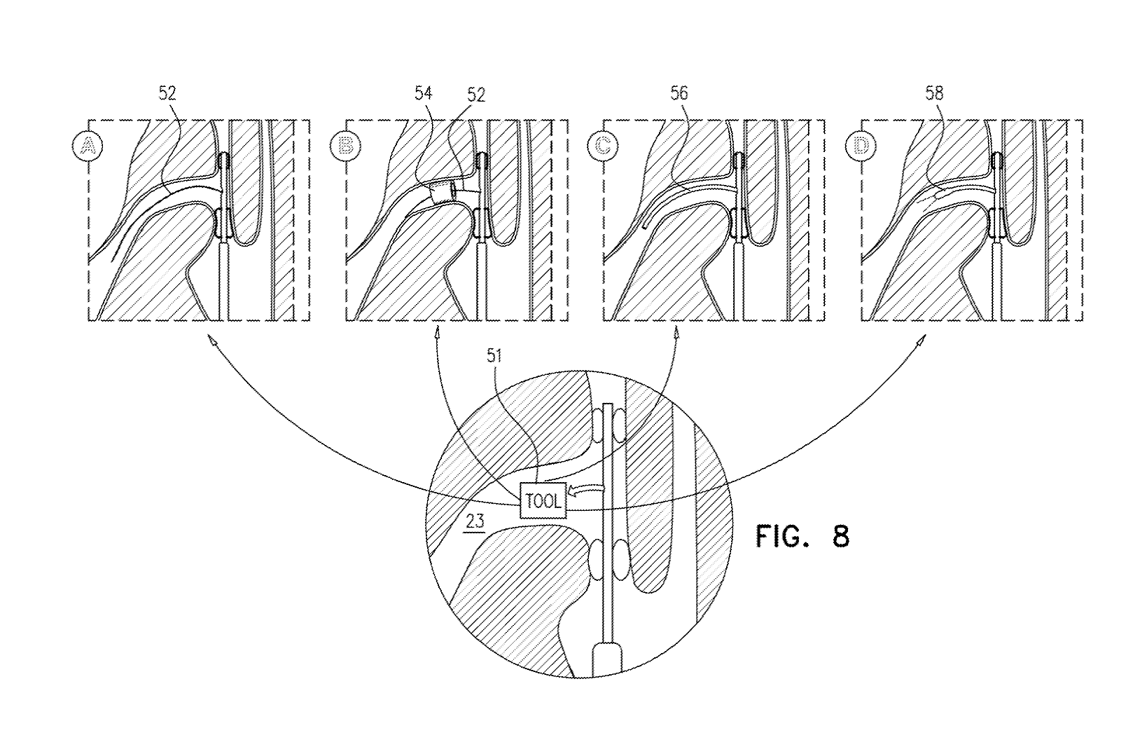

Reference is now made to FIGS. 5A-B, which, show cross-sections of the distal portion of tube 22, in accordance with some applications of the present invention. In some applications, a deflector 38 is disposed within lumen 24 of tube 22, deflector 38 being shaped to deflect out of the aperture a wire that is advanced distally within the lumen of the tube. For example, as shown in FIG. 5A, the deflector may comprise an appendage extending from an inside wall of the tube. Alternatively, as shown in FIG. 5B, the deflector may comprise a curved channel 42. The deflector may facilitate the deployment of a guidewire and/or other tools following the opening of the ostium, as described hereinbelow with reference to FIG. 8.

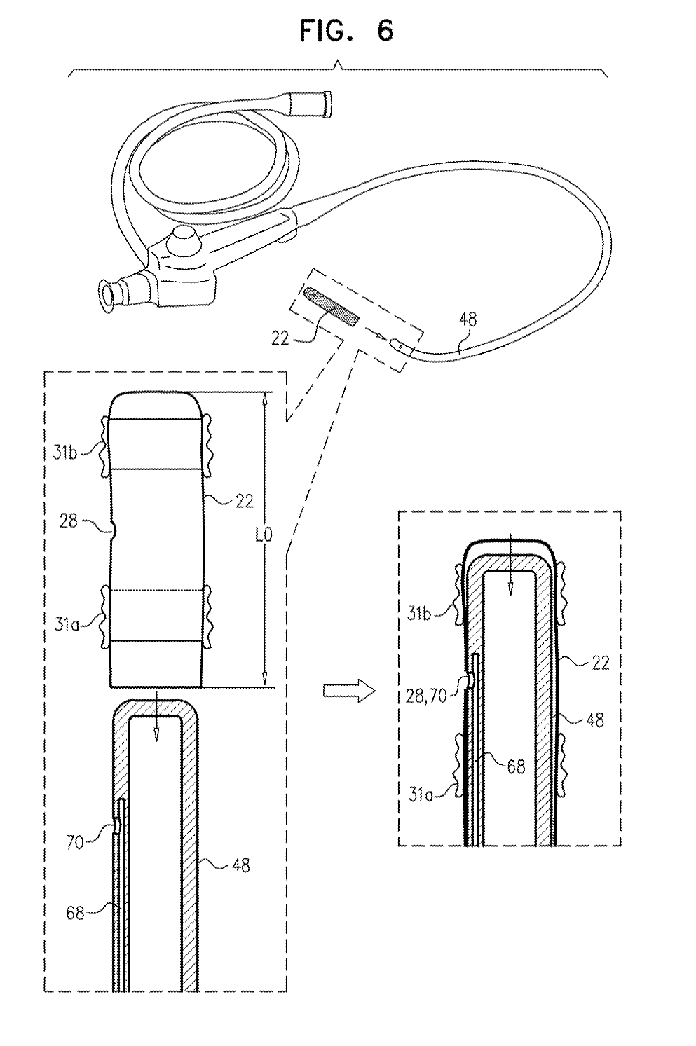

Reference is now made to FIG. 6, which is a schematic illustration of tube 22 placed over the distal end of an endoscope 48, in accordance with some applications of the present invention. In some applications, to facilitate the procedure, tube 22 is placed over the distal end of endoscope 48 prior to being inserted into the subject's nasal cavity. In such applications, length L0 of the tube is typically at least 10 mm and/or less than 30 mm. (Alternatively, L0 may be greater than 30 mm, as described hereinabove with reference to FIG. 2A.) The tube is then inserted into, and moved within, the nasal cavity while surrounding the distal end of the endoscope. Images obtained by the endoscope may be used to guide the advancement of the tube. In some applications, images obtained by the endoscope are also used to guide the subsequent passing of the fluid from the aperture.

In some applications, a fluid-delivery system 68 of the endoscope is used to deliver the fluid to the aperture. As shown in FIG. 6, an opening 70 in the lateral wall of the endoscope may be aligned with aperture 28, such that fluid delivered via fluid-delivery system 68 passes through both opening 70 and aperture 28.) in other applications, before passing the fluid from the aperture, the endoscope is withdrawn, without withdrawing the tube. In such applications, a separate fluid-delivery system (e.g., a fluid-delivery channel or tube running in a proximal-distal direction through the wall of the tube, or lumen 24 of the tube), rather than the fluid-delivery system of the endoscope, is used to deliver fluid to the aperture. One or more inflation tubes 32 (FIG. 2A-B) may run alongside the endoscope, the distal ends of the inflation tubes being in fluid communication with the balloons. Alternatively, the balloons may be inflated via a fluid-delivery system of the endoscope.

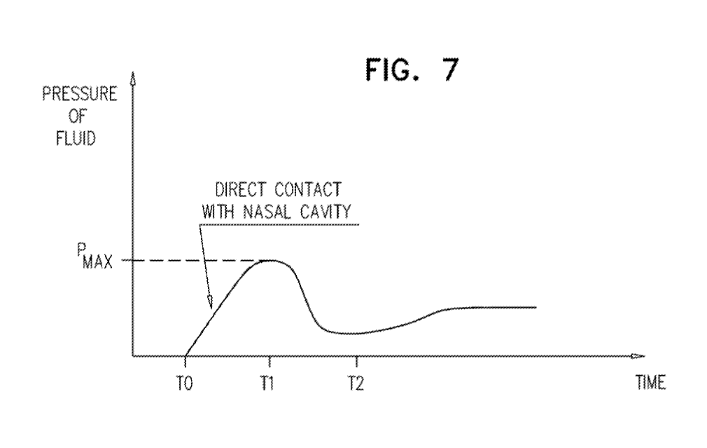

Reference is now made to FIG. 7, which is an illustrative plot showing the pressure of the fluid that is passed from the aperture during the treatment of the natural ostium (as described hereinabove with reference to FIG. 1), in accordance with some applications of the present invention. Starting at time T0, fluid is pumped into the nasal cavity of the subject (e.g., using a syringe) such that the pumped fluid is in direct contact with the nasal cavity. (In particular, as described hereinabove with reference to FIG. 1, fluid is pumped into portion 59 of the nasal cavity.) At time T1, the fluid reaches a maximum pressure PMAX, which is sufficient to open the ostium. The pressure of the fluid then drops rapidly, as the fluid enters the sinus. Subsequently, starting at time T2, the sinus is rinsed, by pumping rinsing fluid into the sinus at a pressure that is less than the maximum pressure.

The scope of the present invention includes various methods of verifying that the ostium has been opened. For example, an imaging device, such as imaging device 44 (FIGS. 2A-B), endoscope 48 (FIG. 6), or an external imaging device may be used to view the ostium while passing the stream of fluid out of the aperture. Upon viewing, using the imaging device, that the ostium has been opened, the physician may stop passing the stream of fluid out of the aperture. Alternatively, as described hereinbelow with reference to FIG. 1, an artificial hole may be formed in the bone wall of the sinus. Upon seeing that the passed fluid is exiting the subject's nose via the artificial hole, the physician may infer that the ostium has been opened, and may therefore, in response, stop passing the fluid. Alternatively, the physician may stop passing the fluid upon feeling a drop in resistance while pushing the fluid from the syringe. In some applications, the physician stops passing the fluid only upon receiving two or more of the indications described above.

In some applications, the sinus is rinsed following the opening of the ostium. Typically, a syringe or electromechanical pump is used to rinse the sinus. In some applications, the syringe or pump is used to alternatingly (i) pump rinsing fluid into the sinus, and (ii) not pump any fluid into the sinus. For example, the syringe or pump may be used to alternatingly (i) pump rinsing fluid into the sinus, and (ii) draw fluid from the sinus. In some applications, the syringe includes a first channel, through which the rinsing fluid is pumped, and a second channel, through which the fluid is released.

Reference is again made to FIG. 1. In some applications, an artificial hole 50 is formed in the bone wall of the sinus, e.g., using techniques described in U.S. application Ser. No. 13/189, 896 to Gross et al., published as U.S. 2013/0030545, which is incorporated herein by reference. Artificial hole 50 facilitates the rinsing of the sinus, by allowing the rinsing fluid to pass out of the sinus therethrough. In addition, as described hereinabove, the artificial hole may help indicate to the physician that the ostium is open, by allowing the passed fluid to exit the subject's nose. Aside from the site for artificial hole 50 shown in FIG. 1, alternate sites along the wall of the sinus may be chosen for the artificial hole, examples of which are indicated by markers M2 and M3 in FIG. 1.

Reference is now made to FIG. 8, which is a schematic illustration showing the passing of a tool 51 into ostium 23, in accordance with some applications of the present invention. In some applications, following the opening of the ostium, a tool 51 is passed through tube 22 and into the ostium. In some applications, (scenario A), the tool includes a guidewire 52. Alternatively or additionally (scenario B), the tool may include a balloon 54 (e.g., a torus-shaped balloon), which may be inflated within the ostium in order to help keep the ostium open. (The balloon may be passed into the ostium via guidewire 52.) Alternatively or additionally (scenario C), the tool may include a rinsing and/or suction tube 56, which may be used to pump rinsing fluid into and/or out of the sinus. In some applications, rinsing and/or suction tube 56 is shaped to define a first tube lumen and a second tube lumen. Fluid is passed through the first tube lumen to rinse the sinus, and back through the second tube lumen to drain the sinus. Alternatively or additionally (scenario D), the tool may include an imaging device 58, which may be used to image the interior of the sinus. Alternatively or additionally (not shown), the tool may include a drill, which may be used to widen the opening leading into the sinus.

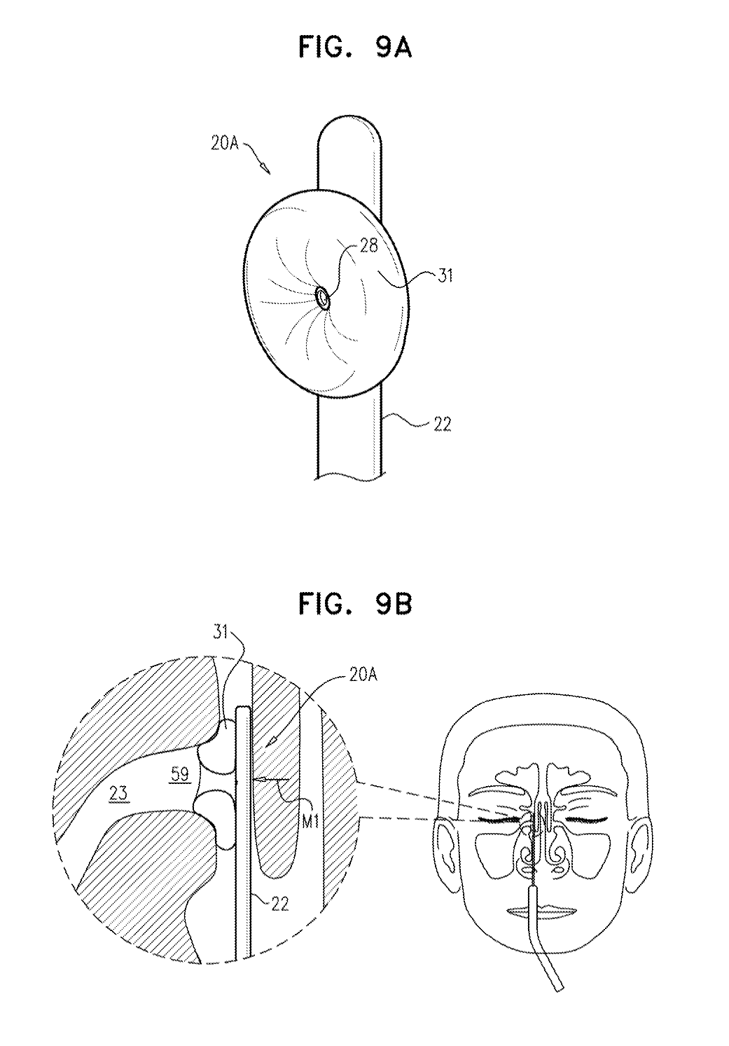

Reference is now made to FIGS. 9A-B, which are schematic illustrations of apparatus 20A for treating a maxillary sinus of a subject, in accordance with some applications of the present invention. Apparatus 20A is generally similar to apparatus 20 as shown in FIGS. 2A-B, except for having exactly one balloon 31 coupled to the tube, rather than two balloons. As shown in FIG. 9B, when inflated inside the nasal cavity, balloon 31 generally fluidly isolates portion 59 of the nasal cavity that is adjacent to the ostium. In other words, balloon 31 generally prevents flow of fluid from portion 59 (a) to the subject's lungs, and (b) to the outer opening of the subject's nostril. Following the inflation of balloon 31, fluid may be passed into fluidly-isolated portion 59, as described hereinabove, in order to open the ostium.

The scope of the present invention includes any appropriate shape for balloon 31, in addition to the shape shown in FIGS. 9A-B. For example, balloon 31 may pass around the full circumference of the tube (but not over the aperture), similarly to balloons 31a and 31b (FIGS. 2A-B). The scope of the present invention further includes applications in which a second balloon is coupled to the tube opposite balloon 31, such that, when the apparatus is deployed as shown in FIG. 9B, the inflation of the second balloon, at approximately the location marked by marker M1, causes the tube (and thus, balloon 31) to foe pushed toward the ostium.

In some applications, a preoperative computed tomography (CT) scan is performed on the subject. In response to anatomical detail shown in the scan, the physician identifies a desired property (e.g., a diameter and/or shape) of one or more of the seals. The physician then selects the seals from a plurality of seals that differ from each other in the particular property. For example, the physician may choose a seal having a particular diameter from a plurality of seals having different respective diameters. Alternatively or additionally, the physician may cause the seals to be manufactured such that the seals have the desired property. Choosing and/or manufacturing the seals in response to the CT scan increases the likelihood that the seals will properly seal the nasal cavity at the desired sites. For example, the diameters and/or shapes of the first and second balloons (FIGS. 2A-B), or the diameter and/or shape of balloon 31 (FIGS. 9A-B), may be matched to the anatomical details that are observed in the scan.

The preoperative scan may also help the physician to properly align the tube during the procedure. For example, the desired insertion depth of the tube may be determined by viewing the preoperative scan. Alternatively or additionally, if the first (proximal) balloon does not inflate to the expected volume, the physician may conclude that the tube has been advanced too far, such that the first balloon is disposed distally to the ostium, where the nasal cavity is narrower. Alternatively or additionally, the alignment of the tube may be facilitated by using an imaging device disposed within the nasal cavity (e.g., imaging device 44, shown, for example, in FIG. 2A), and/or by passing a guidewire out of aperture 28 (e.g., as shown in FIG. 8) and probing the tissue of the nasal cavity with the guidewire. Alternatively or additionally, a radiopaque marker may be placed at or near the ostium, and the alignment of the tube may be subsequently facilitated by use of fluoroscopy.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-24. (canceled)

25. A method for treating a natural ostium of a maxillary sinus of a subject, the method comprising:
opening the ostium, by pumping fluid into a nasal cavity of the subject such that the pumped fluid is in direct contact with the nasal cavity and reaches a maximum pressure; and
subsequently, rinsing the sinus, by pumping rinsing fluid into the sinus at a pressure that is less than the maximum pressure.

26-28. (canceled)

29. The method according to claim 25, further comprising forming an artificial hole in a bone wall of the sinus, wherein rinsing the sinus comprises causing the rinsing fluid to pass out of the sinus through the artificial hole.

30. A method for treating a natural ostium of a maxillary sinus of a subject, the method comprising:
inserting a tube into a nasal cavity of the subject, a wall of the tube at a distal portion of the tube being shaped to define an aperture;
subsequently, moving the tube such that the aperture is aligned generally opposite to the ostium;
subsequently, fluidly isolating a portion of the nasal cavity that is adjacent to the ostium from (a) a lung of the subject, and (b) an outer opening of a nostril of the subject, by deploying one or more seals near the aperture; and
subsequently, opening the ostium, by passing a stream of fluid out of the aperture and into the fluidly-isolated portion of the nasal cavity.

31. The method according to claim 30, further comprising, before inserting the tube:
performing a computed tomography (CT) scan on the subject; in response to anatomical detail shown in the CT scan, identifying a desired property of one or more of the seals; and
causing the seals to be manufactured such that the seals have the desired property.

32. (canceled)

33. The method according to claim 30, further comprising, before inserting the tube:
performing a computed tomography (CT) scan on the subject; and
in response to anatomical detail shown in the CT scan, selecting one or more of the seals from a plurality of seals that differ from each other in a particular property.

34. (canceled)

35. The method according to claim 30, wherein moving the tube such that the aperture is aligned generally opposite to the ostium comprises aligning the aperture with respect to the ostium by using an imaging device disposed within the nasal cavity of the subject.

36. The method according to claim 30, wherein moving the tube such that the aperture is aligned generally opposite to the ostium comprises aligning the aperture with respect to the ostium by (i) passing a guidewire out of the aperture, and (ii) probing tissue of the nasal cavity with the guidewire.

37. The method according to claim 30, wherein deploying the one or more seals comprises deploying one or more annular seals.

38. (canceled)

39. The method according to claim 30, further comprising:
using an imaging device to view the ostium while passing the stream of fluid out of the aperture; and
stopping to pass the stream of fluid, upon viewing, using the imaging device, that the ostium has been opened.

40. The method according to claim 30, further comprising, before inserting the tube into the nasal cavity of the subject, forming an artificial hole in a bone wall of the sinus.

41. (canceled)

42. The method according to claim 30, wherein at least one of the seals includes a balloon, and wherein deploying the seals comprises inflating the balloon.

43. The method according to claim 30, wherein deploying the one or more seals comprises radially deploying (a) a first seal, coupled to the tube proximally to the aperture, and (b) a second seal, coupled to the tube distally to the aperture.

44. The method according to claim 43, wherein radially deploying the first and second seals comprises radially deploying the first and second seals by passing the stream of fluid out of the aperture.

45. The method according to claim 44,
wherein the first seal comprises a first flap,
wherein the second seal comprises a second flap, and
wherein radially deploying the first and second seals comprises radially deploying the first and second flaps by causing the fluid to apply a force to the first and second flaps.

46. The method according to claim 43,
wherein the first seal includes a first balloon,
wherein the second seal includes a second balloon, and
wherein radially deploying the seals comprises inflating the first and second balloons.

47. The method according to claim 46, wherein inflating the first and second balloons comprises (a) inflating the first balloon to a first diameter, and (b) inflating the second balloon to a second diameter that is less than the first diameter.

48. The method according to claim 46, wherein inflating the first and second balloons comprises (a) inflating the first balloon to a first pressure, and (b) inflating the second balloon to a second pressure that is less than the first pressure.

49. The method according to claim 46, wherein inflating the first and second balloons comprises inflating the first and second balloons to a single pressure.

50-53. (canceled)

54. The method according to claim 30, wherein at least one of the seals includes a flap, and wherein deploying the seals comprises radially deploying the flap by uncovering the flap.

55. The method according to claim 30, further comprising, following the opening of the ostium, passing a tool through the tube and into the ostium.

56. (canceled)

57. The method according to claim 55, wherein the tool includes a balloon, the method further comprising inflating the balloon within the ostium.

58. The method according to claim 55, wherein the tool includes a rinsing tube, the method further comprising using the rinsing tube to rinse the sinus.

59. (canceled)

60. The method according to claim 55, wherein the tool includes an imaging device, the method further comprising using the imaging device to image an interior of the sinus.

61. The method according to claim 30,
wherein inserting the tube comprises inserting the tube while the tube surrounds a distal end of an endoscope, and
wherein moving the tube comprises moving the tube while the tube surrounds the distal end of the endoscope.

62-63. (canceled)
